(19) United States
(12) Patent Application Publication
(10) Pub. No.: US 2026/0007793 A1
(43) Pub. Date: Jan. 8, 2026

(54) Sanitizing Methods

(71) Applicant: Grignard Pure LLC, Rahway, NJ (US)

(72) Inventors: Emile Etienne Grignard, III, Rahway, NJ (US); Mark Lojek, Rahway, NJ (US)

(21) Appl. No.: 19/112,039

(22) PCT Filed: Sep. 19, 2023
(86) PCT No.: PCT/US2023/033150

§ 371 Date: Mar. 14, 2025

Related U.S. Application Data
(60) Provisional application No. 63/376,642, filed on Sep. 22, 2022.
(60) Provisional application No. 63/481,725, filed on Jan. 26, 2023.

(51) Int. Cl. A61L 2/22; A01N 25/06; A01N 31/02; A01P 1/00; A61L 2/20; A61L 9/015; A61L 9/14; A61L 101/38

(52) U.S. Cl. CPC A61L 2/22; A01N 25/06; A01N 31/02; A01P 1/00; A61L 2/20; A61L 9/015; A61L 9/14; A61L 2101/38; A61L 2202/11; A61L 2202/25; A61L 2209/135

(57) ABSTRACT

This disclosure relates to a method for sanitizing the air and surfaces in a space. The method includes dispersing a sanitizing composition containing a glycol (e.g., triethylene glycol) into the space at a frequency to maintain the sanitizing composition in an aerosol form at a concentration ranging from about 0.02 mg/m3 to about 0.09 mg/m3 in the space.

### CROSS REFERENCE TO RELATED APPLICATIONS

This application claims the priority benefit of U.S. Provisional Application No. 63/376,642, filed Sep. 22, 2022, and U.S. Provisional Application No. 63/481,725, filed Jan. 26, 2023, the content of each of which is incorporated by reference in its entirety herein.

### TECHNICAL FIELD

This disclosure relates to compositions and methods for sanitizing a space (e.g., an indoor space).

### BACKGROUND OF THE DISCLOSURE

Human disease is frequently caused by pathogenic microorganisms such as bacteria, viruses, and fungi. The movement of an infectious particle from a host or infected individual to a susceptible new victim can occur by various mechanisms, including breathing of aerosolized fluids from the host, contact with surfaces contaminated by the host or host bodily fluids, or by transfer on the hands of the victim or third party from the host or contaminated surfaces to the victim. The particular transfer mechanism depends on the organism as well as the particular setting. For example, in hospitals and other clinical environments, organisms such as Enterococcus faecium, Staphylococcus aureus, Klebsiella pneumoniae, Acinetobacter baumannii, Pseudomonas aeruginosa, and Enterobacter species (collectively known as ESKAPE pathogens) and Clostridium difficile can cause a significant amount of hospital-acquired infections. Additionally, multi-drug resistant organisms (e.g., MRSA), predominantly bacteria, that are resistant to one or more classes of antimicrobial agents, have special clinical significance because of their acquired resistance. Hospital-acquired infections have become a significant problem for the health-care industry. As another example, in the food preparation industry, large-scale food packaging facilities are periodically linked to outbreaks of antibiotic-resistant Salmonella enterica, causing numerous deaths. As a further example, mold or fungi in a space can cause health related issues and damage to properties. Thus, there is a need for improved methods and compositions for sanitizing the space in the health care facilities, food packaging facilities, and other settings.

### SUMMARY OF THE DISCLOSURE

The inventor has surprisingly found that aerosols (e.g., liquid droplets) containing triethylene glycol (TEG) can be maintained in the air in an indoor space at a low concentration, while still be effective in inactivating (e.g., killing) pathogens in the air and/or on the surfaces in the indoor space, thereby effectively sanitizing (e.g., disinfecting or sterilizing) the indoor space (i.e., occupied or unoccupied by human being).

In one aspect, this disclosure features a method for sanitizing a space, the method including dispersing a sanitizing composition comprising triethylene glycol into the space at a selected frequency to maintain the sanitizing composition in an aerosol form at a concentration ranging from about 0.02 mg/m³ to about 0.09 mg/m³ in the space.

Other features, objects, and advantages will be apparent from the description and the claims.

### DETAILED DESCRIPTION OF THE DISCLOSURE

As defined herein, unless otherwise noted, all percentages expressed should be understood to be percentages by weight to the total weight of a composition.

In general, this disclosure relates to compositions and methods for sanitizing a space (e.g., an indoor space) by inactivating (or killing) a pathogen (e.g., a microbe or bacterium, a virus, or a fungus) in the space. Examples of suitable spaces include those in offices, schools, hotels, lobbies, theaters, reception rooms, bathrooms, health care facilities (e.g., nursing homes, hospital rooms (e.g., intensive care facilities), and medical offices (e.g., dental offices)), food packaging facilities, agriculture farming facilities, indoor ranches (e.g., poultry, swine, or cattle ranches), institutional kitchens, cafeterias, restaurants, public transportation vehicles (buses, trains, subways, and airplanes), ambulances, indoor stadiums and athletic facilities, law enforcement facilities (e.g., prisons), government facilities, elevators, retail locations, and other indoor public or private spaces. In general, the sanitizing composition described herein can be used to inactivate a pathogen either in an unoccupied space (e.g., a space not occupied by human) or an occupied space (e.g., a space occupied by human).

As used herein, the term “inactivating a pathogen” refers to killing a pathogen or otherwise make the pathogen inactive. For example, the sanitizing composition described herein can inactivate viruses, kill bacteria, kill mycobacterium, kill spores, kill mold and mildew.

In some embodiments, the pathogens that can be inactivated by the sanitizing composition described herein can include bacteria (e.g., gram-positive bacteria, gram-negative bacteria, or antibiotic-resistant bacteria), fungi (e.g., molds), viruses (e.g., enveloped viruses or non-enveloped viruses), and spores (e.g., spores produced by fungi or bacteria). Examples of pathogens that can be inactivated by the sanitizing composition described herein include Methicillin-resistant Staphylococcus aureus (MRSA; a gram-positive bacterium)), a mold (e.g., an Aspergillus such as Aspergillus brasiliensis), a Pseudomonas (e.g., Pseudomonas aeruginosa; a gram-negative bacterium), a Listeria (e.g., Listeria monocytogenes; a gram-positive bacterium), a Salmonella (e.g., Salmonella enterica; a gram-negative bacterium), a Klebsiella (e.g., Klebsiella pneumonia, a gram-negative bacterium), a Mycobacterium (e.g., Mycobacterium tuberculosis), a spore (e.g., an anthrax spore, a Clostridium difficile spore, or a mold spore), or a mixture thereof. Examples of enveloped viruses that can be inactivated by the sanitizing composition described herein include lassa virus, marburg virus, pneumonia, smallpox, croup virus, human parainfluenza viruses (HPIVs), respiratory syncytial virus (RSV), ebola virus, German measles (rubella), herpes simplex (HSV), mumps, Influenza (e.g., H1N1 virus), coronavirus (e.g., SARA-COV-2 virus), and chickenpox. Examples of non-enveloped viruses that can be inactivated by the sanitizing composition described herein include rhinoviruses, enteroviruses, and parvoviruses.

In some embodiments, this disclosure features a method of sanitizing a space (e.g., an indoor space). In some embodiments, the method can include dispersing (e.g., by dispersing, spraying, nebulizing, atomizing, or vaporizing) a sanitizing composition containing triethylene glycol into a space (e.g., an indoor space) to maintain the sanitizing composition in an aerosol form at a concentration ranging from about 0.02 mg/m³ to about 0.09 mg/m³ in the space, thereby inactivating (e.g., killing) any pathogen in the space (e.g., a pathogen introduced into the space before or after the space is treated by the sanitizing composition). In some embodiments, dispersing the sanitizing composition can be performed by a system that generates an aerosol (either visible or non-visible to human eyes) in a space, such as a vaporizer (e.g., a smoke generator), a nebulizer (e.g., a scent dispersion unit), or an atomizer (e.g., a humidifier). When the aerosol is visible to human eyes, the aerosol can be in the form of a fog, smoke, or haze. The system can be those known in the art, such as the fog/haze machines or smoke simulators used in emergency training or used in the lighting industry to generate theatrical effects.

In some embodiments, the concentration of the sanitizing composition in an aerosol form (e.g., the concentration of the liquid droplets containing the sanitizing composition) in a space can be from at least about 0.02 mg/m³ (e.g., at least about 0.03 mg/m³, at least about 0.04 mg/m³, at least about 0.05 mg/m³, at least about 0.06 mg/m³, at least about 0.07 mg/m³, or at least about 0.08 mg/m³) to at most about 0.09 mg/m³ (e.g., at most about 0.08 mg/m³, at most about 0.07 mg/m³, at most about 0.06 mg/m³, at most about 0.05 mg/m³, at most about 0.04 mg/m³, or at most about 0.03 mg/m³). For example, the sanitizing composition in an aerosol form can have a concentration ranging from about 0.02 mg/m³ to about 0.05 mg/m³ in the space. In general, the aerosolized sanitizing composition in the above concentration range is significantly below the concentration range (i.e., at least about 0.5 mg/m³) that can be detected by human eyes and therefore is non-visible to human eyes. Without wishing to be bound by theory, it was surprisingly discovered that maintaining the sanitizing composition in an aerosol form at a concentration as low as from about 0.02 mg/m³ to about 0.09 mg/m³ is effective in inactivating (or killing) various pathogens in the space (either suspending in the air or on a surface). Further, without wishing to be bound by theory, it was surprisingly discovered that, while maintaining the concentration of the aerosolized sanitizing composition within the concentration range described above (i.e., from about 0.02 mg/m³ to about 0.09 mg/m³) is effective in inactivating (or killing) various pathogens in the space, increasing the concentration of the aerosolized sanitizing composition above this concentration range does not necessarily increase the efficacy of the composition. In addition, without wishing to be bound by theory, it is believed that the aerosolized sanitizing composition can generate a vapor containing a glycol (e.g., triethylene glycol or propylene glycol), which can inactivate a pathogen in the space.

In general, the aerosolized sanitizing composition can form vapor containing TEG and other optional components (e.g., another glycol such as propylene glycol) in the sanitizing composition within the space to be treated. In some embodiments, the total concentration of the TEG (including TEG in the aerosol and TEG in the vapor) or glycol (e.g., including both TEG and another glycol such as propylene glycol) in a space can be from at least about 0.4 mg/m³ (e.g., at least about 0.5 mg/m³, at least about 0.6 mg/m³, at least about 0.8 mg/m³, at least about 1 mg/m³, at least about 1.2 mg/m³, at least about 1.4 mg/m³, at least about 1.5 mg/m³, at least about 1.6 mg/m³, or at least about 1.8 mg/m³) or at most about 2 mg/m³ (e.g., at most about 1.8 mg/m³, at most about 1.6 mg/m³, at most about 1.5 mg/m³, at most about 1.4 mg/m³, at most about 1.2 mg/m³, at most about 1 mg/m³, at most about 0.8 mg/m³, at most about 0.6 mg/m³, or at most about 0.5 mg/m³). For example, the total concentration of the TEG (including TEG in the aerosol and TEG in the vapor) or glycol (including glycol in the aerosol and in the vapor) in a space can be from about 0.4 mg/m³ to about 0.7 mg/m³ (e.g., from about 0.5 mg/m³ to about 0.6 mg/m³).

In some embodiments, to maintain the sanitizing composition in an aerosol form in a space at a desired level (e.g., at a concentration range from about 0.02 mg/m³ to about 0.09 mg/m³), the method described herein can include dispersing the sanitizing composition at a certain frequency, which can depend on various factors, such as the size of the space, the temperature and humidity of the space, the air flow conditions (e.g., the air exchange rate) in the space, the particle size of the aerosolized composition, and the amount of the composition introduced into the space during each dispersing period. In some embodiments, the method described herein can include dispersing the sanitizing composition for a dispersing period (during which a certain amount of the sanitizing composition is introduced into the space) of from at least about 5 seconds (e.g., at least about 10 seconds, at least about 15 seconds, at least about 20 seconds, at least about 25 seconds, or at least about 30 seconds) to at most about 1000 seconds (e.g., at most about 900 seconds, at most about 800 seconds, at most about 700 seconds, at most about 600 seconds, at most about 500 seconds, or at most about 300 seconds), followed by a non-dispersing period (during which no sanitizing composition is introduced into the space) of from about 15 seconds (e.g., at least about 20 seconds, at least about 30 seconds, at least about 40 seconds, at least about 50 seconds, at least about 1 minute, at least about 2 minutes, at least about 4 minutes, or at least about 5 minutes) to about 10 minutes (e.g., at most about 8 minutes, at most about 6 minutes, at most about 5 minutes, at most about 4 minutes, at most about 2 minutes, or at most about 1 minute). In some embodiments, the method described herein can include dispersing the sanitizing composition in an amount of at least about 2 grams (e.g., at least 3 grams, at least about 4 grams, or at least about 5 grams) to at most about 25 grams (e.g., at most about 20 grams, at most about 15 grams, at most about 10 grams, at most about 9 grams, at most about 8 grams, at most about 7 grams, at most about 6 grams, or at most about 5 grams) for a space having a volume of about 1000 cubic feet every 6 hours to keep the aerosolized sanitizing composition at a desired concentration level (e.g., from about 0.02 mg/m³ to about 0.09 mg/m³) during the 6-hour period.

In some embodiments, to maintain the sanitizing composition in an aerosol form in a space at a desired level (e.g., at a concentration range from about 0.02 mg/m³ to about 0.09 mg/m³), the method described herein can include dispersing the sanitizing composition into the space on demand. In such embodiments, the method can include dispersing the sanitizing composition into the space through a system that includes an aerosol generator (e.g., a diffusion device) and a sensor. The aerosol generator can be configured to generate aerosol particles having an appropriate size (such as the particle sizes described herein) such that the aerosol particles can be suspended in the air. The sensor can be in fluid communication with the air within the space to be treated and configured to measure the concentration of the sanitizing composition aerosolized within the space. The measured concentration can be provided on a display on the sensor or provided to a user through a mobile application. In addition, the sensor can be configured to operate the aerosol generator to disperse the sanitizing composition into the space. The concentration of the aerosolized sanitizing composition within the space can be measured by the sensor and operation of the aerosol generator can be controlled to achieve a desired concentration of the aerosolized sanitizing composition within the space. The concentration measuring and dispersing steps can be repeated periodically as needed to maintain the desired concentration of the aerosolized sanitizing composition within the space. Examples of such a system have been described in U.S. Provisional Application No. 63/365,729, the entire contents of which is incorporated herein by reference. In some embodiments, the above method can be automated so that the aerosol concentration can be controlled within a desired range (e.g., from about 0.02 mg/m³ to about 0.09 mg/m³) automatically.

In some embodiments, the sanitizing composition can be in a steady state (i.e., the concentration of the aerosolized sanitizing composition can be maintained in range from about 0.02 mg/m³ to about 0.09 mg/m³) for an extended period of time (e.g., from 5 minutes to 24 hours) per day) within the space to be treated. For example, the sanitizing composition can be maintained in a steady state at a desired concentration for at least about 5 minutes (e.g., at least about 10 minutes, least about 20 minutes, at least about 30 minutes, at least about 1 hour, at least about 2 hours, at least about 3 hours, at least about 4 hours, or at least about 6 hours) to at most about 24 hours (e.g., at most about 18 hours, at most about 15 hours, at most about 12 hours, at most about 10 hours, or at most bout 9 hours) per day. Without wishing to be bound by theory, it is believed that the sanitizing composition described herein can form a saturated vapor in a space at a relatively low aerosol concentration (e.g., from about 0.02 mg/m³ to about 0.09 mg/m³) and that the vapor pressure of the saturated vapor can allow the aerosol particles to form a uniform dispersion throughout the space in a steady state. In general, vapor refers to a material (e.g., the sanitizing composition) in the gas phase at a temperature lower than its critical temperature (e.g., the vapor can be condensed to a liquid by increasing the pressure on it without reducing the temperature of the vapor). Further, in general, aerosol refers to a suspension of liquid droplets (e.g., liquid droplets of the sanitizing composition) in air or another gas. Further, in general, saturated vapor refers to refers to a material (e.g., the sanitizing composition) in the gas phase that would condense to the liquid phase with a small change in pressure and/or temperature.

In some embodiments, the aerosolized sanitizing composition can be maintained at a uniform concentration (i.e., in range from about 0.02 mg/m³ to about 0.09 mg/m³) for an extended period of time (e.g., from 5 minutes to 24 hours)) within the space to be treated.

In some embodiments, the method can further include vaporizing the sanitizing composition (e.g., in an aerosol generator such as a humidifier, a fog/haze machine, a smoke generator, a nebulizer, or an atomizer) before dispersing (e.g., spraying) the composition into a space. In some embodiments, vaporizing the composition can be performed by treating the composition with steam or heating. For example, when vaporizing the composition is performed by heating, the method can include delivering the composition to a heat exchanger to vaporize the composition. The heated vapor can be forced through a nozzle as vapor and/or liquid droplets (or liquid particles) to form a visible or invisible aerosol. For example, when the sanitizing composition described herein is dispersed (e.g., sprayed) into a space using a vaporizer, the vaporizer can have a liquid reservoir and can use a pump (e.g., an electric pump) to propel the sanitizing composition in the liquid reservoir into a heat exchanger where the sanitizing composition is vaporized. The heated vapor is forced through a nozzle as vapor and as liquid droplets (or liquid particles) that form a visible or non-visible aerosol.

In some embodiments, when the sanitizing composition is dispersed (e.g., sprayed) into a space using an atomizer (e.g., a nebulizer), the composition can be converted to an aerosol by pressure. For example, the composition can be pumped or siphoned into a series of specific sized chambers in an atomizer or nebulizer to increase the pressure and velocity of the composition in order to convert it from a liquid to an aerosol. In some embodiments, ultrasonic and/or vibrating mesh technology can be used to disperse the composition into a space through an atomizer or a nebulizer.

In some embodiments, dispersing the sanitizing composition can form vapor and/or liquid droplets (or liquid particles) that contain triethylene glycol (or other glycols). In some embodiments, the liquid droplets can form an aerosol that contains triethylene glycol (or other glycols). In some embodiments, dispersing the composition can form an aerosol, a vapor, or a mixture thereof. In some embodiments, the aerosol liquid droplets can have an average diameter of from at least about 10 nm (e.g., at least about 20 nm, at least about 50 nm, at least about 100 nm, at least about 200 nm, at least about 500 nm, at least about 1 μm, at least about 2 μm, or at least about 5 μm) to at most about 10 μm(e.g., at most about 8 μm, at most about 6 μm, at most about 5 μm, at most about 4 μm, at most about 2 μm, at most about 1 μm, or at most about 0.5 μm). In some embodiments, the method described herein can generate from at least about 2000 (e.g., at least about 3000, at least about 4000, at least about 5000, at least about 6000, at least about 8000, or at least about 10,000) to at most about 150,000 (e.g., at most about 50,000 or at most about 25,000) liquid droplets per cm³ of the space (e.g., the indoor space).

In some embodiments, dispersing (e.g., spraying) the sanitizing composition can be performed intermittently (e.g., either at a constant interval or at irregular intervals). In some embodiments, when the composition is applied intermittently at a constant interval, the frequency of the application can vary as desired depending on factors such as the concentration of triethylene glycol or other glycols in the composition, the temperature and humidity of the space, the size of the space, the desired concentration of the composition in the space, and the air exchange rates. In some embodiments, the preferred temperature of the space can range from about 5° C. to about 50° C. (e.g., from about 10° C. to about 30° C. or from about 15° C. to about 30° C.). In some embodiments, the preferred relative humidity of the space can range from about 5% to about 75% (e.g., from about 15% to about 70%, from about 30% to about 65%, or from about 45% to about 60%). In some embodiments, the time period between two applications of the composition can be from at least about 10 seconds (e.g., at least about 30 seconds, at least about 1 minutes at least about 1.5 minutes, at least about 2 minutes, at least about 5 minutes, at least about 10 minutes, at least about 30 minutes, or at least about 1 hour) to at most about 2 hours (e.g., at most about 1 hour, at most about 30 minutes, at most about 10 minutes, or at most about 5 minutes).

In some embodiments, dispersing (e.g., spraying) the sanitizing composition can be performed continuously. In some embodiments, the sanitizing composition can be dispersed continuously into a space for at least about 5 minutes (e.g., at least about 10 minutes, at least about 30 minutes, at least about 1 hour, or at least about 2 hours) to at most about 24 hours (e.g., at most about 12 hours, at most about 9 hours, at most about 6 hours, at most about 5 hours, at most about 4 hours, at most about 3 hours, at most about 2 hours, or at most about 1 hour).

Without wishing to be bound by theory, it is believed that the sanitizing composition or the TEG having a concentration within the ranges described herein can effectively kill or inactivate at least 95% (e.g., at least about 96%, at least about 97%, at least about 98%, at least 98.5%, at least 99%, at least 99.5, or at least 99.9%) of a pathogen in a space within a short period of time (e.g., at most 60 minutes, at most 30 minutes, at most 15 minutes, at most 10 minutes, at most 5 minutes, at most 3 minutes, at most 2 minutes, at most 1 minute, or at most 30 seconds). In some embodiments, the sanitizing composition or the TEG having a concentration within the ranges described herein may take a relatively long time (e.g., at least about 4 hours, at least about 8 hours, at least about 12 hours, or at least about 24 hours) to reach the above inactivation efficacy (e.g., at least about 95%) for certain pathogens (e.g., a mold) that are known to be difficult to inactivate. In some embodiments, at least about 0.5 gram (e.g., at least about 1 gram, at least about 2 grams, at least about 3 grams, or at least about 4 grams) and/or at most about 10 grams (e.g., at most about 5 grams) of the sanitizing composition can be used in a space having a volume of 1000 cubic feet every 4 hours (e.g., every two hours or every one hour).

In some embodiments, to practice the sanitizing method described herein in an indoor space, one can place a system described herein (e.g., a vaporizer or an atomizer such as a nebulizer) in the center or on one or more sides of the indoor space to be treated. In some embodiments, a system described herein can be incorporated in an HVAC system that controls the temperature, humidity, and/or purity of the air within an indoor space. In such embodiments, the system described herein may not need to be placed in the indoor space to be treated. In some embodiments, multiple systems can be used at appropriate places to ensure even distribution of the sanitizing composition. The sanitizing composition described herein can be applied from the system(s) into the indoor space until a desired sanitization (or disinfection) level is achieved. In some embodiments, the sanitizing composition can be applied continuously or intermittently (e.g., every minute or every 10 minutes) to maintain the desired sanitization level.

In some embodiments, the sanitizing composition described herein can be applied to an indoor space to be treated via an HVAC unit. For example, a system containing the sanitizing composition described herein can be connected to the return plenum of an HVAC unit through a tubing. The composition can then be applied into the indoor space through the HVAC unit until a desired sanitization (or disinfection) level is achieved. This approach can sanitize both the filter in the HVAC unit and the indoor space. In at least some implementation, the composition is not substantially removed from the air by the HVAC unit. For example, in some implementations, at least some of the composition in droplet form may adhere to a filter of the HVAC unit, but may nevertheless vaporize and release from the filter and into the environment.

In some embodiments, the space (e.g., the indoor space) to be treated can include a pathogen suspending in the air and the sanitizing method described herein is capable of inactivating (e.g., killing) the pathogen in the air. In some embodiments, the space can include a pathogen on a surface (e.g., either a hard or soft surface, or either a non-porous or a porous surface) and the sanitizing method described herein is capable of inactivating (e.g., killing) the pathogen on the surface. In some embodiments, the surface can be any surface in an indoor space, such as a surface of a wall, a floor, a desk, a chair, a computer, a rug, a plant, or a drape. Without wishing to be bound by theory, it is believed that triethylene glycol (or other glycol) in the vapor generated from the aerosolized sanitizing composition can adhere to the pathogen either in the air or on a surface to inactivate the pathogen by denaturing or disrupting the protein or membrane of the pathogen. Further, without wishing to be bound by theory, it is believed that, when the pathogen is a bacterium, a mycobacterium, a mold, or a spore, the sanitizing composition described herein can inactivate (e.g., kill) the pathogen by desiccation.

In some embodiments, the sanitizing composition described herein can contain (e.g., comprise, consist essentially of, or consist of) triethylene glycol (or other glycols) and water (e.g., deionized water). Triethylene glycol is miscible with water, has a boiling point of 286.5° C. at a pressure of 101.325 kPa, and has a relative low vapor pressure compared to water. Without wishing to be bound by theory, it is believed that a glycol (e.g., triethylene glycol or propylene glycol) in the vapor generated from the aerosolized sanitizing composition inactivates a pathogen by condensing on a pathogen until the concentration of the glycol becomes sufficiently high to denature the pathogen. Further, without wishing to be bound by theory, it is believed that a glycol (e.g., triethylene glycol or propylene glycol) is highly hydroscopic and can inactivate (e.g., kill) a pathogen by absorbing water from the pathogen. In addition, without wishing to be bound by theory, it is believed that triethylene glycol (or propylene glycol) has very low acute or chronic toxicity when inhaled or ingested (especially at the level used in the air to sanitize (e.g., disinfect) an indoor space) and therefore is safe to use in indoor spaces (occupied or unoccupied).

In general, the amount of the glycol (e.g., triethylene glycol or propylene glycol) in the sanitizing composition described herein is not particular limited and can vary as desired. For example, a sanitizing composition containing a relatively low amount of triethylene glycol can achieve the same disinfection effect as a sanitizing composition containing a relatively high amount of triethylene glycol by applying the former composition in an indoor space at a higher frequency or in a higher amount. In some embodiments, the sanitizing composition described herein can include triethylene glycol in an amount of from at least about 1% (e.g., at least about 5%, at least about 10%, at least about 20%, at least about 30%, at least about 40%, at least about 50%, at least about 52%, at least about 55%, at least about 60%, at least about 65%, at least about 70%, at least about 75%, or at least about 80%) by weight to at most about 99.5% (e.g., at most about 99%, at most about 95%, at most about 90%, at most about 85%, at most about 80%, at most about 75%, at most about 70%, at most about 65%, at most about 60%, or at most about 50%) by weight of the composition. In some embodiments, triethylene glycol can be 100% of the sanitizing composition described herein (i.e., without any other ingredient). It is believed that applying a sanitizing composition containing a relatively high amount (e.g., at least about 50% by weight) of triethylene glycol can increase the efficiency of the disinfection and reduce the frequency of the application of the composition.

In some embodiments, the water in the sanitizing composition described herein is deionized water, reverse osmosis (RO) water, or ultrapure water (e.g., when used in a vaporizer). In some embodiments, the water can have a resistivity of at least 17 mega Ohms, a total organic carbon content of at most about 10 ppb, a bacterial count of at most about 10 CFU/ml). For example, the water can include ions in an amount of from at most about 50 ppm (e.g., at most about 40 ppm, at most about 30 ppm, at most about 20 ppm, at most about 10 ppm, at most about 5 ppm, or at most about 1 ppm) to at least about 1 ppb (e.g., at least about 10 ppb) of the total amount of the water. In some embodiments, when the sanitizing composition described herein is used in connection with an atomizer (e.g., a nebulizer), the water in the composition can be tap water (i.e., not deionized water, RO water, or ultrapure water).

In some embodiments, the sanitizing composition described herein can include water in an amount of from at least about 1% (e.g., at least about 5%, at least about 10%, at least about 15%, at least about 20%, at least about 25%, at least about 30%, at least about 35%, at least about 40%, at least about 45%, at least about 48%, at least about 50%, at least about 60%, or at least about 70%) by weight to at most about 99% (e.g., at most about 95%, at most about 90%, at most about 85%, at most about 80%, at most about 75%, at most about 70%, at most about 65%, at most about 60%, at most about 50%, or at most about 48%) by weight of the composition. Without wishing to be bound by theory, it is believed that using deionized water, RO water, or ultrapure water can minimize clogging the nozzles (e.g., caused by deposition of minerals in water) of the system (e.g., a vaporizer) used to apply the sanitizing composition described herein and therefore can keep the system operating for an extended period of time. In addition, without wishing to be bound by theory, it is believed that the water in the sanitizing composition described herein can facilitate inactivation of pathogens by facilitating the evaporation of the glycol constituents of a sanitizing compound

Without wishing to be bound by theory, it is believed that including water in the sanitizing composition can allow the composition to be readily nebulized, atomized or vaporized (e.g., by reducing the vaporization temperature and/or increasing the evaporation rate when the sanitizing composition is applied by an atomizer such as a nebulizer, a humidifier, a fog/haze machine, or a smoke generator) and to form an aerosol or vapor in the atmosphere. The water in the aerosol can evaporate rapidly to form fine glycol droplets and/or vapor, which have sanitizing effects and inactivate pathogens in the air or on a surface. In addition, the water in the sanitizing composition can render the composition inflammable, thereby resulting in a safer product than TEG alone (which is a flammable liquid having a flash point of 157° C.).

In some embodiments, the sanitizing composition described herein can further include an optional ingredient, such as a glycol different from triethylene glycol. In some embodiments, the additional glycol can be propylene glycol. Without wishing to be bound by theory, it is believed that the additional glycol can increase the sanitizing effect of the composition. In some embodiments, the sanitizing composition described herein does not include any additional glycol or any components other than triethylene glycol and water.

In some embodiments, the sanitizing composition described herein can include an additional glycol (e.g., propylene glycol (PG)) in an amount of from at least about 0.5% (e.g., at least about 1%, at least about 1.5%, at least about 2%, at least about 2.5%, at least about 3%, at least about 3.5%, at least about 4%, at least about 4.5%) by weight to at most about 99% (e.g., at most about 95%, at most about 90%, at most about 80%, at most about 70%, at most about 60%, at most about 50%, at most about 40%, at most about 30%, at most about 20%, at most about 10%, at most about 5%, at most about 4.5%, at most about 4%, at most about 3.5%, at most about 3%, at most about 2.5%, at most about 2%, at most about 1.5%, or at most about 1%) by weight of the composition.

In some embodiments, the sanitizing composition described herein can optionally include a material generally recognized as safe (“GRAS”) as defined by the U.S. Food and Drug Administration. Examples of suitable GRAS materials include dimethyl ether, glycerin, chlorine dioxide, and hypochlorous acid.

In some embodiments, the sanitizing composition described herein can include (e.g., comprise, consist essentially of, or consist of) from about 50% to about 90% by weight triethylene glycol and from about 10% to about 50% by weight water. In some embodiments, the sanitizing composition described herein can include (1) triethylene glycol in an amount of from about 52% to about 90% by weight of the composition; (2) water in an amount of from about 5% to about 48% by weight of the composition; and (3) propylene glycol in an amount of from about 0% to about 5% (e.g., from about 0.5% to about 5%) by weight of the composition. In some embodiments, the sanitizing composition described herein can include about 52.25% by weight triethylene glycol, about 1% by weight propylene glycol, and about 46.75% by weight deionized water.

The following examples are illustrative and not intended to be limiting.

### EXAMPLE 1

### Evaluation of a Sanitizing Composition for its Efficacy Against MS2 Bacteriophage in an Aerosol

Sanitizing Composition #1 was tested for efficacy against MS2 bacteriophage with two different test devices (i.e., Hurricane 1800 Flex (Chauvet DJ, Sunrise, FL) and Amhaze Stadium (Chauvet DJ, Sunrise, FL)) at various airborne concentrations. Sanitizing Composition #1 contained triethylene glycol (52.25 wt %), propylene glycol (1 wt %), and DI water (46.75%).

MS2 Bacteriophage (MS2), ATCC 15597-B1 was selected for this test. This virus is a non-enveloped positive-stranded RNA virus of the bacteriophage family Leviviridae. Bacterial cells are the hosts for bacteriophages, and E. coli 15597 served this purpose for MS2 bacteriophage. Its small size, icosohedral structure, and environmental resistance has made MS2 ideal for use as a surrogate virus.

MS2 was grown on appropriate media. The culture used for test inoculum are evaluated for sterility, washed and concentrated in sterile phosphate buffered saline upon harvesting. Virus concentrations were determined after incubation at 36±1° C. for 18-24 hours. MS2 samples were enumerated in 50% tryptic soy agar using standard dilution and plating techniques. The test inoculum was split into two equal parts and added to the appropriate number of nebulizers. Liquid culture should not exceed 20 ml per nebulizer. The testing parameters are as follows:
Volume of inoculum added to nebulizer: 20 ml
Sampler Media (Volume): Phosphate buffered saline with 0.1% Tween 80 (20 ml)
Sampling Time: 10 minutes
Sampling Type: Impingers, SKC biosamplers
Incubation Temperature: 36±1° C.
Virus Nebulization Time: 60 minutes
Neck Rinse Media (Volume): Phosphate buffered saline (5 ml)
Sanitizing Composition Contact Times: 0, 3 minutes, 15 minutes, and 27 minutes
Enumeration Media: 50% Tryptic Soy Agar
Incubation Time: 18-24 hours
Test Substance:
   Sanitizing Composition #1: triethylene glycol (52.25 wt %), propylene glycol (1 wt %), and DI water (46.75%)
   Number of Samples: 3

The test device was placed on the floor of the testing chamber, in a complete horizontal position at a distance of ˜5 feet from the low level carrier placement. The test device was plugged into the outlet and then the wired timer controller was plugged into the remote connector socket on the test device. The test device was allowed to heat up for three to five minutes. With the fluid intake tube in the test substance bottle, the manual button on the remote control was pressed to prime the machine. The test sanitizing composition was then weighed following the priming and returned to the test device for use in testing.

The testing chamber was setup and the safety checklist was completed prior to test initiation. Tests were initiated by aerosolizing the MS2 using the nebulizers for 60 minutes and allowing the concentration to reach the required amount. Once the target virus concentration is reached, a time zero sample was taken and then the test device was run for the specified contact time and an additional sample was taken for each contact time. In a baseline run, no test composition was added to the testing chamber and samples were taken at 3 minutes, 15 minutes, and 27 minutes to determine virus die off and settling at these times. In a test run, the test device was run for a predetermined time (i.e., 30 seconds for the Amhaze Stadium device and 3 seconds for the Hurricane 1800 Flex device) to add a target amount of Sanitizing Composition #1 to the test chamber and a sample was taken at each contact time (3 minutes, 15 minutes, and 27 minutes). All samples were taken in phosphate buffered saline with 0.1% Tween 80 (20 ml) for 10 minutes by using an SKC Biosampler®. The amounts of Sanitizing Composition #1 used by the Amhaze Stadium device the Hurricane 1800 Flex device were 51 grams and 13 grams, respectively.

Once the test was completed, the decontamination process was performed by using 4 hours of UV exposure prior to any scientist entering the testing chamber. Reductions of MS2 were calculated relative to its concentration at the time zero or corresponding control run sample as applicable.

The viral concentration in the testing chamber was calculated using the following equations:

$$PFU / ml = ( Average plate count ) \times 1 : 10 serial dilution factor$$

$$PFU / {m}^{3} = [ ( PFU / ml \times {V}_{s} ) \div ( {T}_{s} \times 12.5 L / min ) ] \times ( 1000 L / {m}^{3} )$$

in which Vₛ=Bio-sampler volume (ml) and Tₛ=Time sampled (min). In addition, the Log10 reduction and percent reduction in the viral concentration are calculated by the following equation:

$$Percent Reduction = [ ( B - A ) / B ] \times 100 \%$$

$${Log}_{10} Reduction = Log ( B / A )$$

$${Log}_{10} Reduction Compared to Baseline = {Log}_{10} Reduction of test - {Log}_{10} Reduction of baseline$$

in which B=Number of viable test microorganisms at time zero after nebulization; and A=Number of viable test microorganisms after the contact time.

The test results are summarized in Table 1 below.

TABLE 1

| | | | | | | Percentage | Log₁₀ | Percent |
|---|---|---|---|---|---|---|---|---|
| | | | | | | Reduction | Reduction | Reduction |
| | | Treatment | | | | Compared | Compared | Compared |
| Test | | Time | | | Average | to Time | to | to |
| Microorganism | Run Type | Point | Replicate | PFU/m³ | PFU/m³ | Zero | Baseline | Baseline |
| MS2 | Baseline | Time | Replicate | 3.30E+05 | 4.06E+05 | N/A | | |
| Bacteriophage | | Zero | 1 | | | | | |
| ATCC 15597- | | | Replicate | 4.83E+05 | | | | |
| B1 | | | 2 | | | | | |
| | | | Replicate | 4.26E+05 | | | | |
| | | | 3 | | | | | |
| | | 3 | Replicate | 4.14E+05 | 3.42E+05 | 15.81% | N/A | N/A |
| | | minutes | 1 | | | | | |
| | | | Replicate | 2.70E+05 | | | | |
| | | | 2 | | | | | |
| | | | Replicate | 2.83E+05 | | | | |
| | | | 3 | | | | | |
| | | 15 | Replicate | 1.53E+05 | 1.66E+05 | 59.22% | N/A | N/A |
| | | minutes | 1 | | | | | |
| | | | Replicate | 1.78E+05 | | | | |
| | | | 2 | | | | | |
| | | | Replicate | 1.07E+05 | | | | |
| | | | 3 | | | | | |
| | | 27 | Replicate | 7.84E+04 | 8.16E+04 | 79.90% | N/A | N/A |
| | | minutes | 1 | | | | | |
| | | | Replicate | 8.48E+04 | | | | |
| | | | 2 | | | | | |
| | | | Replicate | 5.06E+04 | | | | |
| | | | 3 | | | | | |
| | Hurricane | Time | Replicate | 3.05E+05 | 3.39E+05 | N/A | | |
| | 1800 | Zero | 1 | | | | | |
| | Flex | | Replicate | 3.73E+05 | | | | |
| | | | 2 | | | | | |
| | | | Replicate | 7.39E+05 | | | | |
| | | | 3 | | | | | |
| | | 3 | Replicate | 8.14E+02 | 8.10E+02 | 99.76% | 2.55 | 99.76% |
| | | minutes | 1 | | | | | |
| | | | Replicate | 8.06E+02 | | | | |
| | | | 2 | | | | | |
| | | | Replicate | 1.78E+02 | | | | |
| | | | 3 | | | | | |
| | | 15 | Replicate | 9.28E+01 | <1.35E+02 | >99.96% | >3.01 | >99.92% |
| | | minutes | 1 | | | | | |
| | | | Replicate | 1.78E+02 | | | | |
| | | | 2 | | | | | |
| | | | Replicate | <8.72E+01 | | | | |
| | | | 3 | | | | | |
| | | 27 | Replicate | <9.12E+01 | <9.08E+01 | >99.97% | >2.88 | >99.89% |
| | | minutes | 1 | | | | | |
| | | | Replicate | <9.04E+01 | | | | |
| | | | 2 | | | | | |
| | | | Replicate | <9.12E+01 | | | | |
| | | | 3 | | | | | |
| | Amhaze | Time | Replicate | 7.46E+05 | 7.56E+05 | N/A | | |
| | Stadium | Zero | 1 | | | | | |
| | | | Replicate | 7.66E+05 | | | | |
| | | | 2 | | | | | |
| | | | Replicate | 4.79E+05 | | | | |
| | | | 3 | | | | | |
| | | 3 | Replicate | 4.83E+03 | 5.35E+03 | >99.29% | 2.08 | 98.44% |
| | | minutes | 1 | | | | | |
| | | | Replicate | 5.86E+03 | | | | |
| | | | 2 | | | | | |
| | | | Replicate | 5.85E+03 | | | | |
| | | | 3 | | | | | |
| | | 15 | Replicate | 1.81E+03 | 1.80E+03 | >99.76% | 2.23 | 98.92% |
| | | minutes | 1 | | | | | |
| | | | Replicate | 1.79E+03 | | | | |
| | | | 2 | | | | | |
| | | | Replicate | 1.72E+03 | | | | |
| | | | 3 | | | | | |
| | | 27 | Replicate | 8.28E+02 | <1.05E+03 | >99.86% | >2.16 | >98.71% |
| | | minutes | 1 | | | | | |
| | | | Replicate | 1.27E+02 | | | | |
| | | | 2 | | | | | |
| | | | Replicate | <9.12E+01 | | | | |
| | | | 3 | | | | | |

As shown in Table 1, the test involving the Amhaze Stadium device (which utilized 51 grams of Sanitizing Composition #1) showed a net percent reduction of 98.44%, 98.92% and at least 98.71% at 3 minutes, 15 minutes and 27 minutes respectively. In addition, the test involving the Hurricane 1800 Flex device (which utilized 13 grams of Sanitizing Composition #1) achieved a net percent reduction of 99.76%, at least 99.92% and at least 99.89% at 3 minutes, 15 minutes and 27 minutes respectively.

### EXAMPLE 2

### Evaluation of a Sanitizing Composition for its Efficacy at a Reduced Amount Against MS2 Bacteriophage in an Aerosol

Sanitizing Composition #1 was tested for efficacy at a reduced amount against MS2 bacteriophage using a GLP protocol with Amhaze Stadium (Chauvet D J, Sunrise, FL)).

The test procedures are described below.

### Processing Glassware

Specialty glassware required for aerosol testing including nebulizers and SKC biosamplers were rinsed with tap water and then deionized water. The glassware was carefully wrapped in a microfiber or other soft material, placed into sterilization bags, and autoclaved. The glassware was allowed to dry at room temperature or at elevated temperatures. The drying conditions were recorded at the time of the study. The glassware was allowed to equilibrate to room temperature prior to use in the study.

### Aerosol Chamber Setup

The aerosol testing chamber including the walls, glove ports, and sampling locations were cleaned with 1:100 diluted household bleach. The disinfectant solution was recorded at the time of the study. Cleaning occurred within 5 days of test initiation and between testing runs. After cleaning, the surfaces were wiped with sterile deionized water and the surfaces were allowed to dry completely. On each day of testing and prior to each run, the biosampler ports were wiped with 1:100 diluted household bleach sprayed onto a microfiber cloth. The disinfectant solution was recorded at the time of the study. The ports and grommets were wiped with the disinfectant solution thoroughly and allowed to dry completely.

### Inoculum Preparation

For MS2 Bacteriophage ATCC 15597-B1: Prepared viral stocks are stored at −70° C.±10° C. until ready to be used for testing. Frozen stocks were removed from freezer on the day of testing and allowed to thaw. Host Culture was grown in 10 ml of tryptic soy broth at 36±1° C. for 6-24 hours. Inoculum was prepared by diluting in phosphate buffered saline to a target concentration of ≥1.0×10⁷ PFU/ml. The inoculum was enumerated. Nebulizers were prepared by adding 15-20 ml of prepared inoculum inside a biological safety cabinet and then replaced the lid on the nebulizers before being transferred to the chamber.

### Aerosol Efficacy Chamber Runs

Four chamber runs were performed. One was a baseline run that was used to establish the natural settling and die-off of the test microorganism after nebulization. Three test runs were performed where the chamber was treated with each of the test substance lots using the test device.

The safety checklist was completed including placement of back-up power, verifying HEPA filter functionality, checking if the UV lights were working properly, covering outlets, initiating pressurized air, verifying sampler pumps, and checking the pressure for the inner chamber. When the safety and pre-study check list was completed to the point of adding nebulizers, fitted N95 masks were donned by the participating analysts. Then the nebulizers were placed and connected to the pressurized air. The mobile UV bulb was placed into the room and the inner chamber was exited and the door was closed and sealed. Once nebulization had been initiated, the door was not re-opened until decontamination occurred.

Prior to the start of nebulization, temperature and humidity was recorded. The pressurized air was initiated and the nebulizers were confirmed to be running by visually observing if all six jets were running in both nebulizers. The nebulizers were allowed to run for 60 minutes±30 seconds. Temperature and humidity were recorded once nebulization has concluded.

The amount of Sanitizing Composition #1 loaded into the device was weighed and recorded. The test device was operated until Sanitizing Composition #1 was observed to be coming out of the device. Priming the device was done outside the chamber for ≥5 minutes. The weight of Sanitizing Composition #1 used during priming was recorded. Test device was then set up and operated by an operator. The operator recorded any data or device settings that pertains to the dosage of Sanitizing Composition #1 into the chamber. Sanitizing Composition #1 was added into the test chamber throughout the test run to achieve and maintain a preset aerosol concentration. The weight of Sanitizing Composition #1 used during testing was recorded.

### Aerosol Collection

Using biosamplers, samples were taken at intervals including a time zero after nebulization and then after predetermined exposure times. The samplers were allowed to run for 10 minutes±10 seconds. The vacuum pumps maintained a pressure of 10-20 psi to run the samplers at 12.5 L/min per the manufacturer's recommendations. The samples were collected in triple replicate at the following sample times after treatment time had begun; Time Zero, 30 seconds, 15 minute, 60 minutes. During the sampling times, the samplers were verified to be sampling appropriately by observing the vortex created within the samplers.

### Processing Biosamplers

Once the samples were collected, the samplers were moved to a biological safety cabinet for processing. Each neck was rinsed with 5 ml of sterile phosphate buffered saline. The liquid was allowed to drain into the collection cup of the vessel. The liquid was transferred into a sterile 50 ml conical or equivalent sterile vessel. During the transfer, the total liquid volume was observed and recorded. The sample was then enumerated. All culture and collected samples were enumerated per ml. The samples were diluted in phosphate buffered saline in 1:10 dilutions as necessary to observe a countable range of plaques. For this assay, the countable range was considered 25-250 colonies per plate. For dilution series with only counts <25 on the least diluted plates, counts less than 25 may then be used in calculations. For dilution series with no counts, the limit of detection was calculated and this may be used to estimate log reductions. It is preferable to use counts within 25-250, however counts outside this range may be used as well. For counts >250, it was noted as too numerous to count (TNTC). The dilutions were plated using pour plate techniques.

MS2 bacteriophage ATCC 15597-B1 was plated to 50% TSA which was supplemented with 0.100 ml per plate with E. coli ATCC 15597. Plates were swirled well to mix and then allowed to solidify prior to incubation.

### Aerosol Chamber Decontamination

All UV lights equipped in the aerosol chamber to facilitate decontamination were activated and allowed to run for a minimum of 2 hours. The air compressor was disconnected and stored appropriately.

### Neutralization Verification Chamber Run

To evaluate the neutralization of the test substances, a neutralization verification chamber run was performed on one representative lot of Sanitizing Composition #1. Each combination of test microorganism and Sanitizing Composition #1 was evaluated for neutralization. A set of safety checks were performed and documented at the time of the test. Once the safety and pre-study checklist was completed to the point of adding nebulizers, fitted N95 masks were donned by the participating analysts. The mobile UV bulb was placed into the room. The amount of Sanitizing Composition #1 loaded into the device was weighed and recorded. The test device was primed until Sanitizing Composition #1 was observed to be coming out of the device. Priming the device was done outside the chamber for ≥5 minutes. The chamber was treated with Sanitizing Composition #1. To simulate test conditions, Sanitizing Composition #1 was released into the chamber by using the test device. Using an SKC Biosampler filled with 20 ml of neutralizer (PBS w/0.001% Tween 80), the pumps were turned on and an air sample was collected for 10 minutes±10 seconds. The vacuum pumps maintained a pressure of 10-20 psi to run the samplers at 12.5 L/min per the manufacturer's recommendations. During the sampling time, the samplers were verified to be sampling appropriately by observing the vortex created within the samplers. The SKC biosampler was then transferred to a biological safety cabinet and was processed as outlined in the Possessing Biosamplers section above.

Three analyses were prepared to ensure that equivalent level of microorganism were recovered from an inert control suspension (PBS), a suspension of neutralizer alone, and a suspension of neutralized Sanitizing Composition #1 recovered from the air as described above. The respective tubes described above were evaluated at equivalent volumes wherein they were inoculated with 10-100 PFU of test microorganism, vortex mixed, and then 1.0 ml aliquots were plated in duplicate. Multiple tubes of the three liquid types (PBS, neutralizer, and neutralized Sanitizing Composition #1) were inoculated to ensure the target concentration of microorganisms was met. Multiple dilutions were performed, and all data was recorded and included in the final report.

### Enumeration of Air Samples

Enumeration plates and controls for MS2 Bacteriophage ATCC 15597-B1 were incubated at 36° C.±1° C. for 18-24 hours.

### Sterility Controls

A 1.0 ml aliquot of the neutralizer was plated on each day of use. A 1.0 ml aliquot of phosphate buffered saline was plated on each day of use. A plate containing only the growth media was incubated alongside the test. A purity streak of the host microorganism was performed on each day of testing. An infectivity control was performed wherein a dilution of the test Inoculum was plated with the host microorganism to confirm infectivity. All sterility controls were incubated alongside the test plates.

The test results were calculated using the same equations outlined in Example 1 and are summarized in Table 2 below.

TABLE 2

| | | | | | | Percentage | Log₁₀ | Log₁₀ |
|---|---|---|---|---|---|---|---|---|
| | | | | | | Reduction | Reduction | Reduction |
| | | Treatment | | | | Compared | Compared | Compared |
| Test | Run | Time | | | Average | to Time | to Time | to |
| Microorganism | Type | Point | Replicate | PFU/m³ | PFU/m³ | Zero | Zero | Baseline |
| MS2 | Baseline | Time | Replicate | 2.66E+08 | 3.80E+08 | N/A | | |
| Bacteriophage | | Zero | 1 | | | | | |
| ATCC 15597- | | | Replicate | 6.13E+08 | | | | |
| B1 | | | 2 | | | | | |
| | | | Replicate | 2.61E+08 | | | | |
| | | | 3 | | | | | |
| | | 30 | Replicate | 2.38E+08 | 4.13E+08 | No | No | N/A |
| | | seconds | 1 | | | reduction | reduction | |
| | | | Replicate | 5.39E+08 | | | | |
| | | | 2 | | | | | |
| | | | Replicate | 4.62E+08 | | | | |
| | | | 3 | | | | | |
| | | 15 | Replicate | 1.75E+08 | 1.72E+08 | 54.62% | 0.34 | N/A |
| | | minutes | 1 | | | | | |
| | | | Replicate | 1.92E+08 | | | | |
| | | | 2 | | | | | |
| | | | Replicate | 1.51E+08 | | | | |
| | | | 3 | | | | | |
| | | 60 | Replicate | 5.93E+07 | 4.25E+07 | 88.82% | 0.95 | N/A |
| | | minutes | 1 | | | | | |
| | | | Replicate | 4.92E+07 | | | | |
| | | | 2 | | | | | |
| | | | Replicate | 1.89E+07 | | | | |
| | | | 3 | | | | | |
| | Test | Time | Replicate | 1.21E+08 | 1.36E+08 | N/A | | |
| | Run #1 | Zero | 1 | | | | | |
| | | | Replicate | 1.58E+08 | | | | |
| | | | 2 | | | | | |
| | | | Replicate | 1.27E+08 | | | | |
| | | | 3 | | | | | |
| | | 30 | Replicate | 4.48E+05 | 5.67E+05 | 99.58% | 2.38 | 2.38 |
| | | seconds | 1 | | | | | |
| | | | Replicate | 7.10E+05 | | | | |
| | | | 2 | | | | | |
| | | | Replicate | 5.42E+05 | | | | |
| | | | 3 | | | | | |
| | | 15 | Replicate | 1.04E+05 | 1.05E+05 | 99.92% | 3.11 | 2.77 |
| | | minutes | 1 | | | | | |
| | | | Replicate | 9.91E+04 | | | | |
| | | | 2 | | | | | |
| | | | Replicate | 1.11E+05 | | | | |
| | | | 3 | | | | | |
| | | 60 | Replicate | 1.16E+04 | 9.81E+03 | 99.993% | 4.14 | 3.19 |
| | | minutes | 1 | | | | | |
| | | | Replicate | 7.74E+03 | | | | |
| | | | 2 | | | | | |
| | | | Replicate | 1.01E+04 | | | | |
| | | | 3 | | | | | |
| | Test | Time | Replicate | 8.14E+07 | 4.51E+07 | N/A | | |
| | Run #2 | Zero | 1 | | | | | |
| | | | Replicate | 4.88E+07 | | | | |
| | | | 2 | | | | | |
| | | | Replicate | 5.15E+06 | | | | |
| | | | 3 | | | | | |
| | | 30 | Replicate | 1.77E+05 | 2.12E+05 | 99.53% | 2.33 | 2.33 |
| | | seconds | 1 | | | | | |
| | | | Replicate | 2.28E+05 | | | | |
| | | | 2 | | | | | |
| | | | Replicate | 2.32E+05 | | | | |
| | | | 3 | | | | | |
| | | 15 | Replicate | 7.76E+04 | 1.01E+05 | 99.78% | 2.65 | 2.30 |
| | | minutes | 1 | | | | | |
| | | | Replicate | 1.10E+05 | | | | |
| | | | 2 | | | | | |
| | | | Replicate | 1.17E+05 | | | | |
| | | | 3 | | | | | |
| | | 60 | Replicate | 1.00E+04 | 1.23E+04 | 99.97% | 3.57 | 2.61 |
| | | minutes | 1 | | | | | |
| | | | Replicate | 1.38E+04 | | | | |
| | | | 2 | | | | | |
| | | | Replicate | 1.30E+04 | | | | |
| | | | 3 | | | | | |
| | Test | Time | Replicate | 2.65E+07 | 1.98E+07 | N/A | | |
| | Run #3 | Zero | 1 | | | | | |
| | | | Replicate | 1.47E+07 | | | | |
| | | | 2 | | | | | |
| | | | Replicate | 1.83E+07 | | | | |
| | | | 3 | | | | | |
| | | 30 | Replicate | 1.09E+05 | 1.01E+05 | 99.49% | 2.29 | 2.29 |
| | | seconds | 1 | | | | | |
| | | | Replicate | 1.09E+05 | | | | |
| | | | 2 | | | | | |
| | | | Replicate | 8.48E+04 | | | | |
| | | | 3 | | | | | |
| | | 15 | Replicate | 5.53E+04 | 6.10E+04 | 99.69% | 2.51 | 2.17 |
| | | minutes | 1 | | | | | |
| | | | Replicate | 6.75E+04 | | | | |
| | | | 2 | | | | | |
| | | | Replicate | 6.02E+04 | | | | |
| | | | 3 | | | | | |

In this example, Sanitizing Composition #1 was intermittently added to the test chamber to achieve and maintain a target aerosol concentration of 0.437 mg/m³. The test was conducted in triplicate and the average weight of Sanitizing Composition #1 utilized during each test was 2.75 grams. As shown in Table 2 above, an average percentage reduction compared to time zero of 99.53%, 99.80% and 99.98% were achieved at 30 seconds, 15 minutes and 60 minutes respectively.

### EXAMPLE 3

### Evaluation of a Sanitizing Composition for its Efficacy at a Reduced Amount Against MS2 Bacteriophage in an Aerosol

Sanitizing Composition #1 was tested for efficacy at a reduced amount against MS2 bacteriophage using a controlled release method with an Aura Nebulizer (Air Essentials, Miami, FL)). The test conditions and parameters are summarized below.
Test Substance: Sanitizing Composition #1.
Test Substance Aerosol Concentration: about 0.04 mg/m³.
Test Organism: MS2 bacteriophage.
Nebulization stock concentration: 7.0×10¹⁰ pfu/ml.
Nebulization Time: 20 minutes.
Chamber Size: 16 m³.
Exposure Temperature: 22-24° C.
Exposure Humidity: 30-40% Relative Humidity.
Contact Time: The contact time for this study was assessed as the amount of time of aerosol contact.
Aerosol Sampling Duration: 10 minutes.
Aerosol Sampling Time Points: 0-minute, 15-minute, 30-minute, 45-minute, 60-minute, 90-minute.
Settling Slide Duration: 90 minutes.
Neutralizer: Phosphate buffered saline solution+0.05% Tween 80.
Agar Plate Medium: Tryptic Soy Agar.

### Test Method

The test procedures are described below.

### Chamber Preparation

Prior to the initiation of testing, and between each trial, the chamber was wiped clean with 95% Isopropyl alcohol and a non-fibrous towel. All ports were cleaned with non-fibrous brushes. All surfaces were allowed to dry before the trial commencement. The lot number of the cleaning solution, as well as the time and date of the cleaning, were recorded for each cleaning of the chamber.

### Preparation of Test Organism

10 mL of previously prepared MS2 bacteriophage (ATCC #15597-B1) stock was removed from the −80° C. freezer and allowed to thaw. The freezer stock used in this study was characterized as >1.0×10¹¹ pfu/ml. After thawing, the freezer stock was diluted 1:4 with phosphate buffered saline solution for testing. 50 mL of this prepared test inoculum was placed into the nebulizer cup of the Collison 24-jet nebulizer for testing. 5 mL of the host culture, E. coli ATCC #15597, was prepared in tryptic soy broth in a 15 mL conical tube and allowed to grow overnight in an incubator at 35±2° C. This host bacteria were used for plating of the bacteriophage after sampling.

### Neutralization Verification

Neutralization verification testing was run to assure that all kill assessed to the test device was due to kill in the air and not kill in the neutralizer PBS solution after sampling and during plating.

For neutralization verification testing, the amount of Sanitizing Composition #1 was calculated based on a 10-minute sample at the predetermined concentration of 0.04 mg/m³, with an impinger pulling 12.5 l/min. To verify that any fluctuations of that concentration wouldn't cause an issue, the verification also occurred at 1 log (10%) and 2 log (100%) higher concentrations than estimated in the chamber. These concentrations of Sanitizing Composition #1 were diluted in PBS+0.005% Tween 80. After thawing MS2 bacteriophage from freezer stock, it was diluted to a concentration below 1.00×10³ pfu/mL and added to each conical tube. The contact time in the PBS mixtures for testing was 15 minutes. After 15 minutes, all samples were diluted, plated, incubated, and enumerated for concentration. The concentration of each sample was compared to that of the control with only PBS solution and a control with the sample diluted in sterile deionized water to confirm neutralization.

### Chamber Testing

Bioaerosol testing for Sanitizing Composition #1 against the MS2 bacteriophage consisted of a single control and a single trial. The control was run to account for any natural die off of the organism airborne over time in the chamber and to establish a non-treated baseline concentration to compare with the trial concentrations.

Along with the bioaerosol aspect of testing, blank glass slides were placed in the chamber to assess the amount of settling yielded by the microorganisms. The 1″×2″ glass slides were placed on a table located 4 feet away from the Aura dispersion device. The Aura reservoir was filled with Sanitizing Composition #1 in preparation for testing. The Aura was adjusted to the settings determined in the concentration matching section of the protocol. The device was then primed before being placed into the chamber. The priming consisted of running the device outside of the chamber for 20 minutes. This 20-minute prime ensured that the lines of the device were full of fresh Sanitizing Composition #1 and that the device was running correctly.

Prior to the introduction of bioaerosol, the device was placed in the center of the chamber on a stainless-steel table and 3 feet off the floor of the chamber. The device was checked to ensure the proper settings were still enabled before the door of the chamber was closed. After the chamber door was locked, all outlets of the chamber were checked to make sure they were sealed, all vacuum pumps were checked to assure they were working, and the pressurized air was verified as functioning. After the checks were complete, the device was switched on remotely and Sanitizing Composition #1 was dispersed into the chamber air for 30 minutes to maintain a preset aerosol concentration prior to the introduction of the test bioaerosol. The 30 minutes served as a pre-conditioning for the room to simulate an environment already having been treated with Sanitizing Composition #1. The nebulization stock was nebulized at 40 psi for a period of 20 minutes prior to testing. After the nebulization of the bioaerosol into the chamber air, the chamber remained sealed until the decontamination and evacuation of the chamber was complete. The temperature and humidity were recorded for the duration of every test.

### Aerosol Collection

All samples were collected using AGI-30 impingers filled with 20 mL of sterile PBS with the addition of 0.005% Tween 80. The addition of tween 80 was used in order to increase the impinger collection efficiency and for the de-agglomeration of all microorganisms. The impingers sampled from opposite corners of the chamber simultaneously. After sampling, the impingers were pooled and collected in conical tubes for plating. The impingers sampled the chamber at 12.5 l/min using suction from the vacuum pumps. Each impinger used in the study was flow characterized prior to the initiation of the study. Aerosol samples were collected at 0-minute, 15-minute, 30-minute, 45-minute, and 60-minute time points. All samples collected were for a duration of 10 minutes.

### Settling Plate Collection

The settling plates were removed from the chamber after the completion of the trial and evacuation of the chamber. Each plate was placed in separate individually labeled 50 mL conical tubes, containing 10 mL of sterile phosphate buffered saline solution+tween 80, to be plated for concentration. Prior to plating, each conical tube was vortexed for 2 minutes and then vigorously shaken for another minute. The samples were then plated using the technique described in the Sample Plating section below for concentration determination.

### Sampler Cleaning

Between each sample, the impingers were submerged in a 10% bleach solution and then in DI water. They were then washed with high pressure tap water in a sink for a total of six times followed by a final DI water rinse. The impingers were then air dried before being used for another sample.

### Sample Plating

For sample plating, the overnight host bacterium (E. coli ATCC #15597) was diluted 1:9 in PBS. 1.5 mL micro centrifuge tubes were filled with 800 μL of sterile PBS and 100 μL of diluted host bacteria stock from the incubator. The number of tubes needed was based on the number of samples to be plated and the predicted dilution ranges needed to plate the samples. Using the set of dilution tubes, serial dilutions were performed with the test samples. For this study, a standard small drop plaque assay technique was instituted for plating. The countable range for this assay was 5-50 plaques. Counts >50 pfu were labeled as too numerous to count (TNTC). The samples were plated in their respective dilution ranges in triplicate on pre-labeled TSA culture plates. The plates were allowed to dry, then placed in the incubator set at 35±2° C. for 24 hours.

### Incubation and Observation

Plates were allowed to incubate at 35±2° C. for 20-24 hours before being removed for enumeration. Plate counts were recorded in lab notebooks at the time of enumeration and transferred to Microsoft Excel spreadsheets for analysis.

### Study Controls

### Control Count

The MS2 bacteriophage nebulization inoculum yielded acceptable starting concentration counts of >1.0×10⁹ pfu/m³.

### Sterility Control

The media sterility check revealed no growth of a contaminant.

### Culture Purity

The host culture showed no signs of contamination when being streak plated on a tryptic soy agar plate.

### Neutralization Control

Neutralizer yielded no signs of contaminant growth after being plated.

### Data Analysis

### Calculation of % Reduction

The starting bioaerosol concentration of the test trial was compared to the starting concentration of the control trial to calculate the effect of the pretreatment of the chamber by Sanitizing Composition #1. The method for nebulization and neb stock remained the same from the control trial to the test trial so the starting bioaerosol concentration of the test trial would be expected to be the same as the control. The change in the starting bioaerosol concentration seen in the test trial was therefore attributed to the pretreatment of the chamber.

To determine the log reduction of the trial at each time point, the percentage of viable virus remaining airborne in the chamber was compared to the initial bioaerosol concentration of the uninhibited control trial. This remaining percentage was then multiplied by the log10 function to yield log reduction.

For net reduction, the natural losses from the control trial were subtracted from the test trial to account for natural losses not caused by Sanitizing Composition #1.

The settling plates from the control trial were directly compared to the settling plates from the test trial to assess reduction. The difference remaining between the control trial and the test trial was multiplied by the log10 function to yield the log reduction. Since the natural die off should be the same between the two trials, the reduction was assessed as net reduction.

For a viable impinge, the aerosol concentration collection (Cₐ) was calculated as follows:

$${C}_{a} = ( {C}_{imp} \times {I}_{vol} \times t ) / {Q}_{imp}$$

in which viable aerosol concentration collection (Cₐ) is cfu or pfu/L of chamber air; viable Impinger concentration collection (C_{Imp}) is cfu or pfu/ml from enumeration of impinger sample; impinger sample collection volume (Iᵥₒₗ) is 20 ml collection fluid/impinge; impinger sample flow rate (Qᵢₘₚ) is 12.5 L/min; and impinger sample time (t) is 5 or 10 minutes, test dependent.

The aerosol system viable delivery efficiency (expressed as %) is calculated as follows:

$$Efficiency = ( {C}_{a} / {V}_{p} ) \times 100 \%$$

in which Vp is viable particles per liter of air in the chamber. All trial results were averaged to obtain a mean and standard deviation for each trial set. Total viable biological concentrations, along with reductions over time, were represented by Log reductions of aerosolized biologicals and were calculated for each trial.

The test results were calculated using the same equations outlined in Example 1 and are summarized in Table 3 below.

TABLE 3

| | | | | | | Net LOG₁₀ | |
|---|---|---|---|---|---|---|---|
| | Control | | | | Control | Reduction | |
| Time | Conc | Trial Conc | | LOG₁₀ | LOG₁₀ | Compared to | Net % |
| (min) | (pfu/m³) | (pfu/m³) | % reduction | Reduction | Reduction | Baseline | Reduction |
| 0 | 1.87E+10 | 4.16E+08 | 97.7714% | 1.65 | 0.00 | 1.65 | 99.7714% |
| 15 | 7.31E+09 | 1.81E+07 | 99.9029% | 3.01 | 0.41 | 2.61 | 99.7524% |
| 30 | 4.19E+09 | 4.37E+06 | 99.9766% | 3.63 | 0.65 | 2.98 | 99.8957% |
| 60 | 2.00E+09 | 1.81E+06 | 99.9903% | 4.01 | 0.97 | 3.04 | 99.9095% |
| 90 | 1.47E+09 | 7.15E+05 | 99.9962% | 4.42 | 1.10 | 3.31 | 99.9514% |

As shown in Table 6, maintaining Sanitizing Composition #1 at a very low aerosol concentration (about 0.04 mg/m³) in the air unexpectedly exhibited average net Log₁₀ pathogen reduction of 2.61 after an exposure time of 15-25 minutes against MS2 bacteriophage in the aerosol. In other words, the composition was able to inactivate more than 99% of MS2 bacteriophage in the aerosol in only 15-25 minutes. The above results suggest that Sanitizing Composition #1 would be effective in inactivating MS2 bacteriophage in the aerosol form in a space.

In addition, the settling test showed that the average concentrations of MS2 bacteriophage settling on a glass slide were 3.02E+06 pfu/slide in the control trial (where Sanitizing Composition #1 was not introduced into the test chamber) and 6.37E+02 pfu/slide in the test trial (where Sanitizing Composition #1 was introduced into the test chamber), respectively. In other words, the results showed that Sanitizing Composition #1 was able to inactivate 99.98% (i.e., a Log₁₀ pathogen reduction of 3.68) MS2 bacteriophage in the air over a 90-minute duration. The above results also suggest that Sanitizing Composition #1 would be effective in inactivating pathogenic MS2 bacteriophage in the air.

Other embodiments are in the following claims.

1. A method for sanitizing an indoor space, comprising:
dispersing a sanitizing composition comprising triethylene glycol into the space at a selected frequency to maintain the sanitizing composition in an aerosol form at a concentration ranging from about 0.02 mg/m³ to about 0.09 mg/m³ in the space.

2. The method of claim 1, wherein the method comprises dispersing the sanitizing composition for a dispersing period of from about 5 seconds to about 1000 seconds, followed by a non-dispersing period of from about 15 seconds to about 10 minutes.

3. The method of claim 1, wherein the total amount of the triethylene glycol in the space is maintained at a concentration ranging from about 0.4 mg/m³ to about 2 mg/m³.

4. The method of claim 1, wherein the sanitizing composition in an aerosol form has a concentration ranging from about 0.02 mg/m³ to about 0.05 mg/m³ in the space.

5. The method of claim 1, wherein the sanitizing composition is in a steady state in the space.

6. The method of claim 1, wherein the method forms the sanitizing composition in the space that is non-visible to human eyes.

7. The method of claim 1, wherein the triethylene glycol is in an amount of from about 10% to about 90% by weight of the sanitizing composition.

8. The method of claim 1, wherein the sanitizing composition further comprises water.

9. The method of claim 8, wherein the water is in an amount of from about 5% to about 90% by weight of the sanitizing composition.

10. The method of claim 1, wherein the sanitizing composition further comprises propylene glycol.

11. The method of claim 10, wherein the propylene glycol is in an amount of from about 0.5% to about 20% by weight of the sanitizing composition.

12. The method of claim 1, wherein the sanitizing composition comprises from about 10% to about 90% by weight triethylene glycol, and from about 10% to about 90% by weight water.

13. The method of claim 1, wherein the sanitizing composition comprises about 52.25% by weight triethylene glycol, about 1% by weight propylene glycol, and about 46.75% by weight water.

14. The method of claim 1, wherein dispersing the sanitizing composition forms a mixture comprising an aerosol and a vapor.

15. The method of claim 14, wherein the aerosol comprises liquid droplets having an average diameter of from about 10 nm to about 10 μm.

16. The method of claim 1, wherein the method is performed by an atomizer or a vaporizer.

17. The method of claim 1, wherein the selected frequency is determined based on sensor data representing a concentration of the sanitizing composition in the space.

18. The method of claim 1, wherein the selected frequency is a predetermined frequency.
